(19) United States
(12) Patent Application Publication
(10) Pub. No.: US 2008/0206149 A1
(43) Pub. Date: Aug. 28, 2008

(54) METHOD, DYE AND MEDICAMENT FOR STAINING THE INTERNAL LIMITING MEMBRANE, EPIRETINAL MEMBRANE, THE VITREOUS AND/OR THE CAPSULE OF AN EYE

(75) Inventors: Christos HARITOGLOU, Munchen (DE); Wolfgang FREYER, Berlin (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignees: LUDWIG-MAXIMILIANS-UNIVERSITAT MUNCHEN, Munchen (DE); FORSCHUNGSVERBUND BERLIN E.V., Berlin (DE)

(21) Appl. No.: 11/871,804

(22) Filed: Oct. 12, 2007

Related U.S. Application Data
(63) Continuation-in-part of application No. PCT/EP2006/005675, filed on Jun. 13, 2006.

(30) Foreign Application Priority Data
Jun. 17, 2005 (EP) 05013151.5

(51) Int. Cl. A61K 49/00

(52) U.S. Cl. 424/9.6

(57) ABSTRACT

The present invention concerns a method of staining the internal limiting membrane, the vitreous and/or the lens capsule of the eye as well as dyes and medicaments suitable for this method. Such dyes, medicaments and methods are needed in ophthalmic surgery, in particular in macular and/or cataract surgery and/or vitrectomy.

### CROSS-REFERENCE TO RELATED APPLICATIONS

This application is a continuation-in-part of International Patent Application No. PCT/EP2006/005675, filed Jun. 13, 2006, which claims priority to European Patent Application No. 05013151.5, filed Jun. 17, 2005, both of which are hereby incorporated by reference.

The present invention concerns a method of staining the internal limiting membrane, epiretinal membranes, the vitreous and/or the lens capsule of the eye as well as dyes and medicaments suitable for this method. Such dyes, medicaments and methods are needed in ophthalmic surgery, in particular in macular and/or cataract surgery and/or vitrectomy.

At present, vital dyes are used to assist ophthalmic surgery both in the anterior as well as in the posterior segment. Especially in macular surgery, dye-assisted vitrectomy allowed a better intraoperative visualization of the vitreoretinal interface. Two dyes are commonly used in ophthalmic surgery:

Trypan blue was first suggested to stain the lens capsule to assist capsulorrhexis and for the evaluation of the corneal endothelium of donor tissue before performing penetrating keratoplasty. It is now also available for staining of epiretinal tissue and the internal limiting membrane (ILM) in macular pucker surgery. While toxic effects were described in an animal model, no dye related complications have been described in humans. Nevertheless, carcinogenic and teratogenic properties of trypan blue have been described in animal models.

Indocyanine green ICG has a long history as a diagnostic tool for the imaging of choroidal perfusion during angiography. Intraocularly, it was used to stain the lens capsule in mature cataract. Recently, ICG was the first dye introduced for ILM staining during macular surgery. However, the known light absorbing and photooxidative properties of ICG made this dye also applicable for the destruction of tumor cells in vivo and in vitro and for photodynamic therapy at the level of the choriocapillaris. While the intravenous application of ICG is still considered very safe, concerns of dye related toxicity when applied intraocularly emerged after several reports on adverse effects observed experimentally and clinically.

It is thus the aim of the present invention to provide dyes which differentially stain the internal limiting membrane, epiretinal membranes, the vitreous and/or the lens capsule of the eye and have only a small or no toxic effect at all on the retinal pigment epithelium (RPE). Further, a medicament containing these dyes, their use and a method of staining the internal limiting membrane, epiretinal membranes, the vitreous and/or the lens capsule should be provided.

This problem is solved by embodiments of the method, dyes, and medicament of the present invention. Further improvements of said method, dyes, medicaments and use are provided in some embodiments of the present invention.

The inventive dyes all stain lens capsules, the vitreous as well as epiretinal membranes in a differential manner thus allowing to differentiate the lens capsule, the vitreous and/or the epiretinal membrane from surrounding tissue, e.g. during macular pucker surgery. All these dyes show a long wavelength absorption maximum within the range of 586 to 634 nm lying in the visible region. Most important and different to the dyes used in the prior art and others not claimed, these dyes showed no relevant toxicity or no toxicity at all, making them suitable for staining tissue in the eye.

The invention thus identifies dyes with satisfying staining characteristics and revealing no relevant or no detectable toxicity.

The inventive dyes light green SF, E68, Bromophenol blue and Chicago blue are defined as follows.

Light green SF:

C₃₇H₃₄N₂Na₂O₉S₃

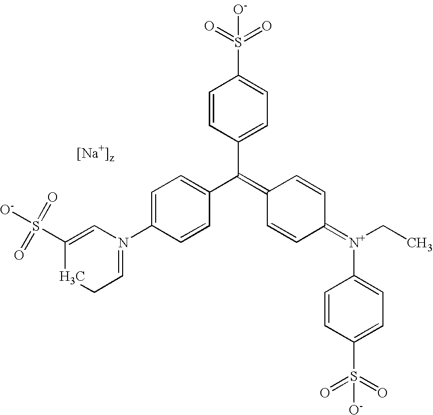

E68:

Copperphtalocyanine-3,4′,4″,4′″-tetrasulphonic acid tetrasodium salt

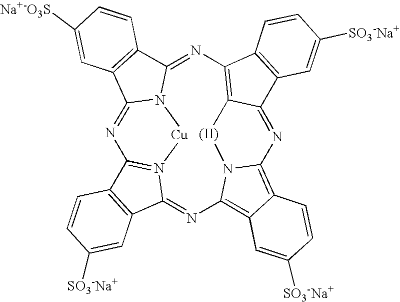

Bromophenole blue:

C₁₉H₁₀Br₄O₅S

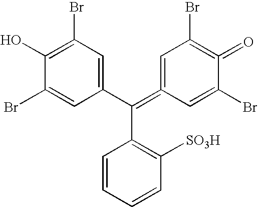

Chicago blue:

C₃₄H₂₄N₆Na₄O₁₆S₄

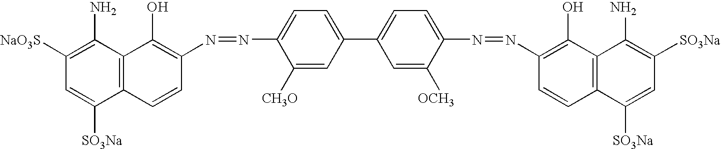

In the following results are shown on the use of the dyes light green SF (LGSF), E68, Bromophenole blue (BPB) and Chicago blue (CB).

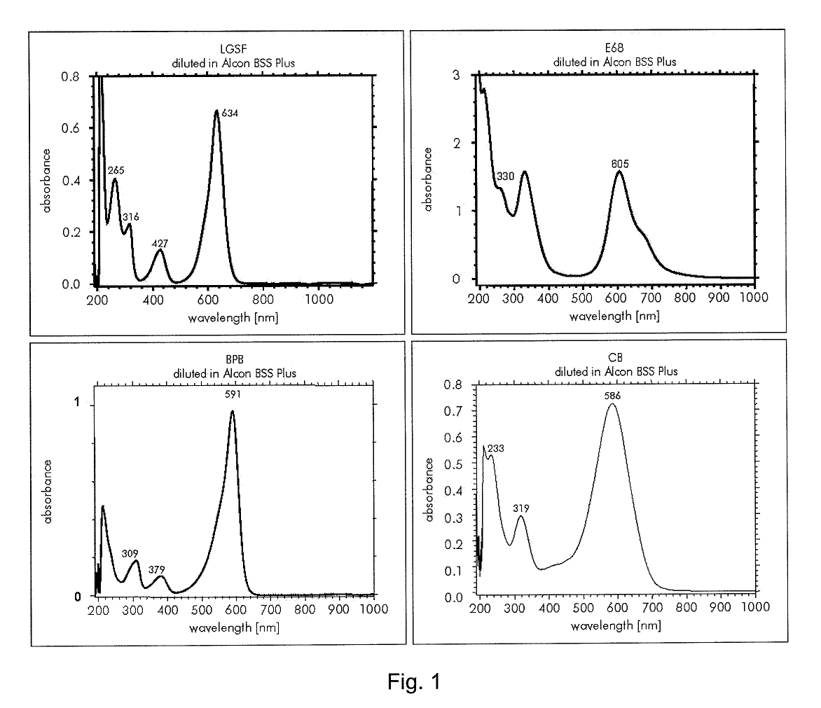

FIG. 1 shows the light absorbing properties of Light green yellowish SF (LG SF), E68, Bromophenol blue (BPB) and Chicago blue (CB). The maximum peak of absorption varied between 586 and 634 nm. Most dyes showed no relevant light absorption between 400 and 500 nm and beyond 700 nm,

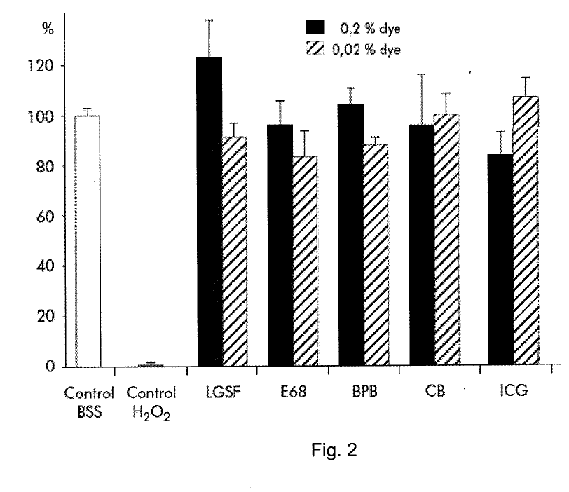

FIG. 2 shows the viability of ARPE-19 cells measured after treatment with the investigated dyes measured by a calorimetric test (MTT). Tests were performed in triplicate and repeated three times. ARPE-19 cells of the same passage incubated with equal volumes of BSS without addition of dyes served as the control. Results were expressed as the mean percentage of control survival. Data are the mean of results in three experiments, each performed in triplicate. Error bars, SEM. (Light green yellowish SF (LG SF), E68, Bromophenol blue (BPB), Chicago blue (CB)). Indocyanine green (ICG) as a commercially available dye was also tested. No statistically relevant differences between both concentrations (0.2% and 0.02%) were observed. BSS plus alone and hydrogenperoxide (H₂O₂,/200 μl/ml) served as controls.

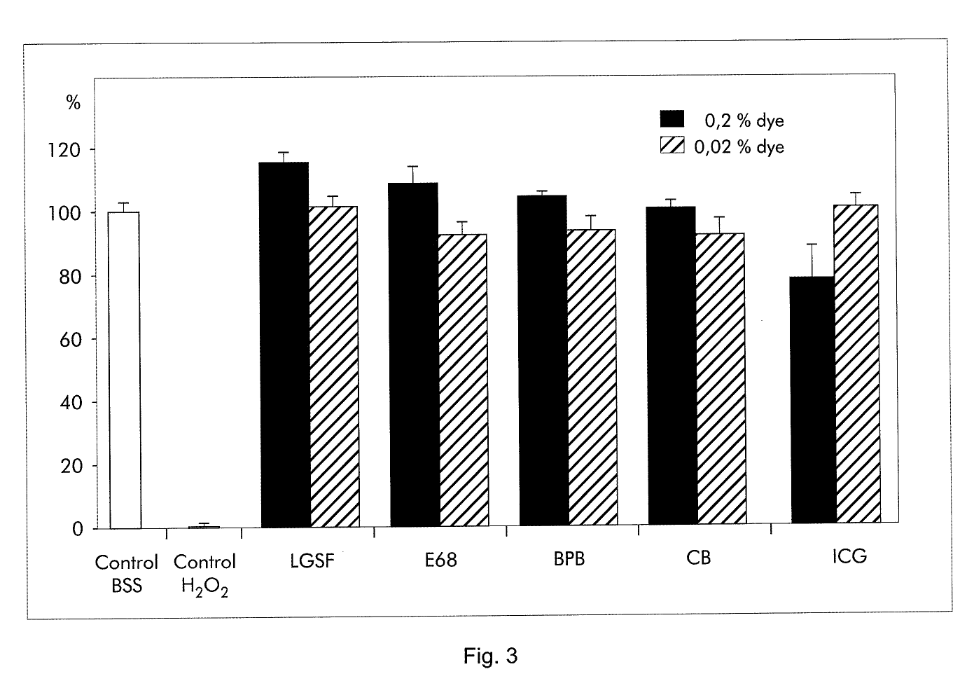

FIG. 3 shows the viability of retinal pigment epithelium (RPE) cells measured after treatment of cells with the investigated dyes measured by a calorimetric test (MTT). RPE cells of the same passage incubated with equal volumes of BSS without addition of dyes served as the control. Results were expressed as the mean percentage of control survival. Data are the mean of results in three experiments, each performed in triplicate. Error bars, SEM. (Light green yellowish SF (LG SF), E68, Bromophenol blue (BPB), Chicago blue (CB)). Indocyanine green (ICG) as a commercially available dye was also tested. The differences between both concentrations were statistically significant only for Light green yellowish SF (LG SF, p≦0.05) and Indocyanine green (ICG, p≦0.05). BSS plus alone and hydrogenperoxide (H₂O₂,/200 μl/ml) served as controls.

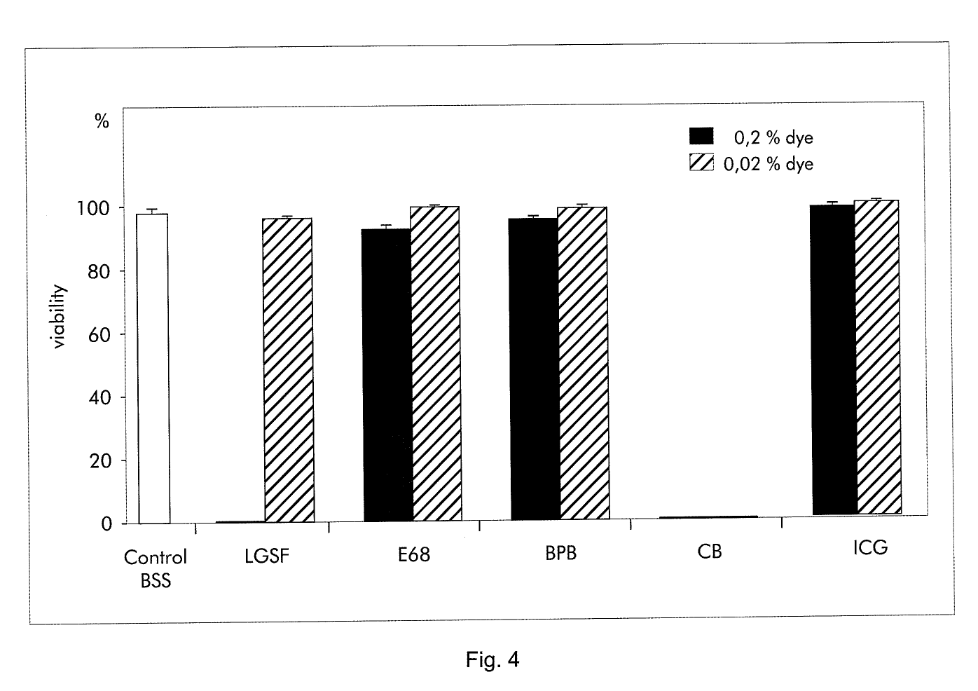

FIG. 4 shows the quantification of the effect of the tested dyes (0.2% and 0.02%) on the number of nonviable cells in cultures of ARPE 19 cells. The percentage of dead cells was scored by counting at least 1400 cells in fluorescence photomicrographs of representative fields. Data (mean±SEM) are based on the sampling of 6 to 10 photomicrographs per condition in three independent experiments performed in duplicate. (Light green yellowish SF (LG SF), E68, Bromophenol blue (BPB), Chicago blue (CB)). Indocyanine green (ICG, 0.5% and 0.05%) as a commercially available dye was also tested.

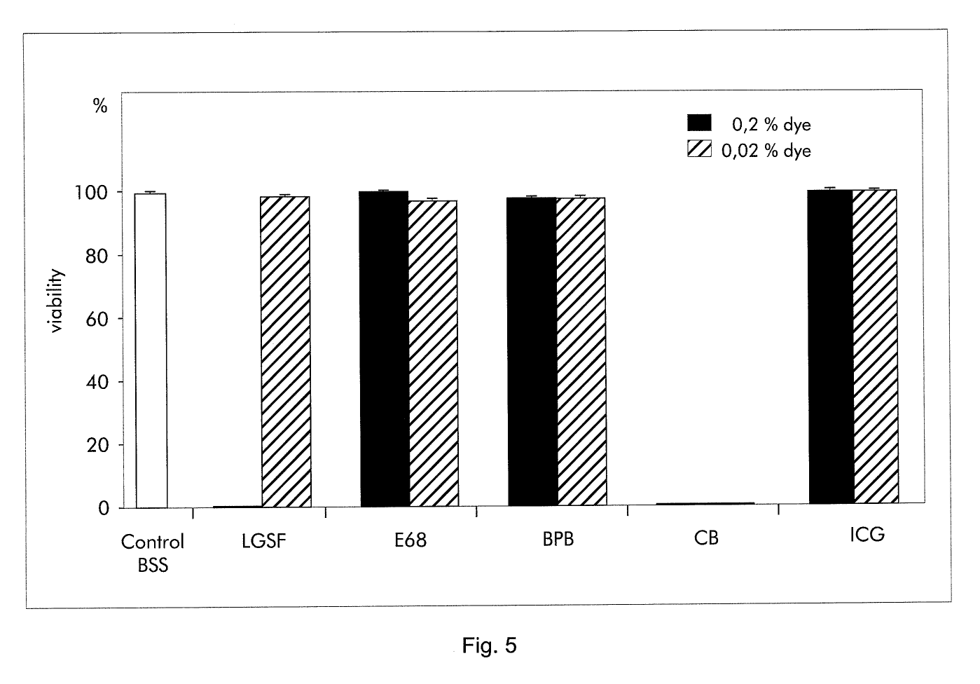

FIG. 5 shows the quantification of the effect of the tested dyes (0.2% and 0.02%) on the number of nonviable cells in cultures of primary RPE cells. The percentage of dead cells was scored by counting at least 1400 cells in fluorescence photomicrographs of representative fields. Data (mean±SEM) are based on the sampling of 6 to 10 photomicrographs per condition in three independent experiments performed in duplicate. (Light green yellowish SF (LG SF), E68, Bromophenol blue (BPB), Chicago blue (CB)). Indocyanine green (ICG, 0.5% and 0.05%) as a commercially available dye was also tested.

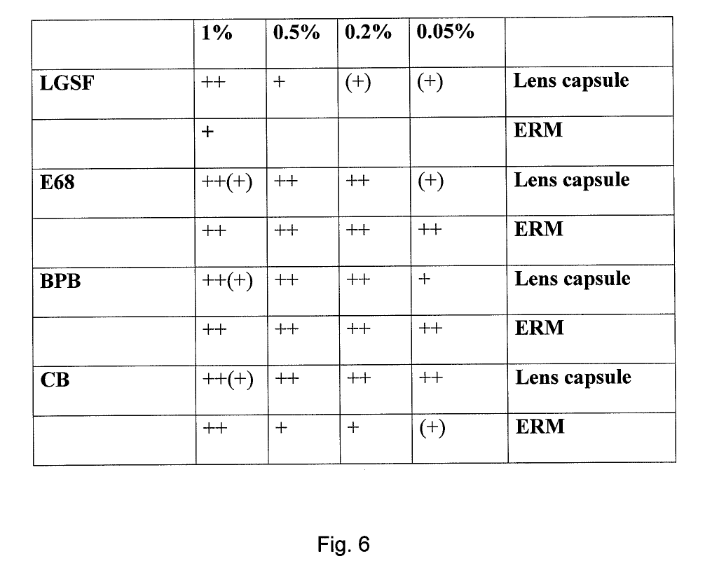

FIG. 6 shows the results of a first example of the grading of the staining effects in lens capsule and epiretinal membrane (ERM) using different dyes and dye concentrations. The staining effects was graded as excellent (+++), good (++), fair (+), absent (−). Light green yellowish SF (LG SF), E68, Bromophenol blue (BPB) and Chicago blue (CB). The first row refers to the results of staining of lens capsule, the second of epiretinal tissue. The staining effect was evaluated against a white background.

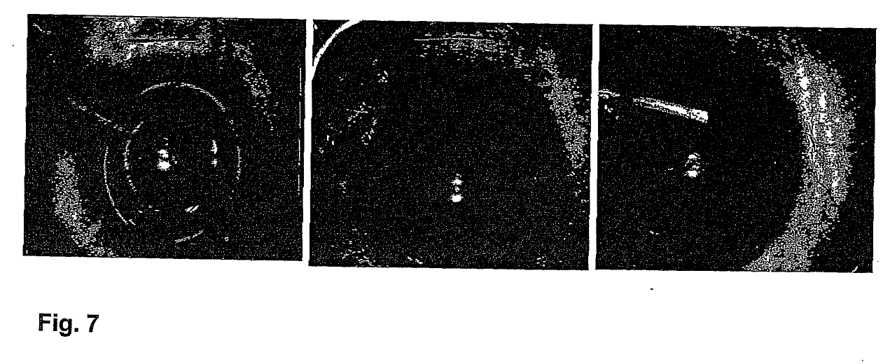

FIG. 7 shows the staining of a lens capsule in a second example. Top: The dye is injected into the air-filled anterior chamber. Middle: After injection of viscoelastic material, a strong and evenly distributed staining of the anterior lens capsule is observed. Bottom: A circular capsulorhexis could be performed showing the contrast between the stained lens capsule and the white cataract.

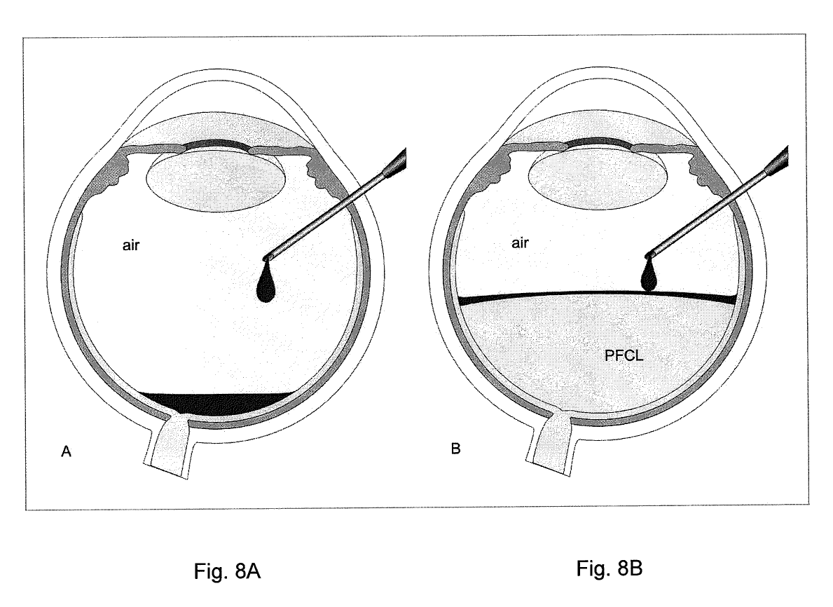

FIG. 8 shows the method of dye injection in a third example: A: During macular hole surgery, the dye is injected into the air-filled globe. B: During vitrectomy for retinal detachment, the globe was first partially filled with perfluorcarbon (PFCL) and fluid (BSS)-air-exchange was performed. The dye was then injected onto the PFCL bubble. With this approach, a higher dye concentration in the anterior segment of the eye could be obtained, an uncontrolled distribution of the dye in the subretinal space as well as an unwanted staining of the lens capsule could be prevented.

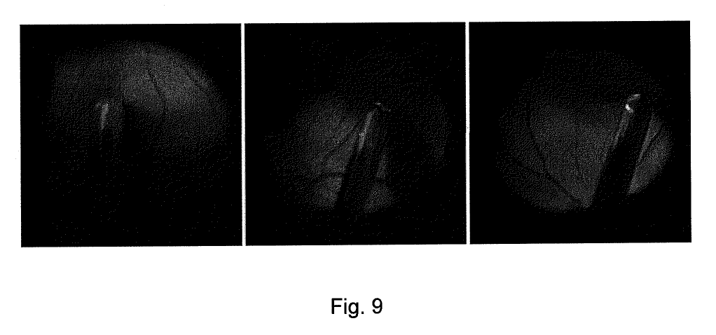

FIG. 9 shows the visualization of a very thin layer of remnant vitreous cortex following dye injection after assumed induction of PVD.

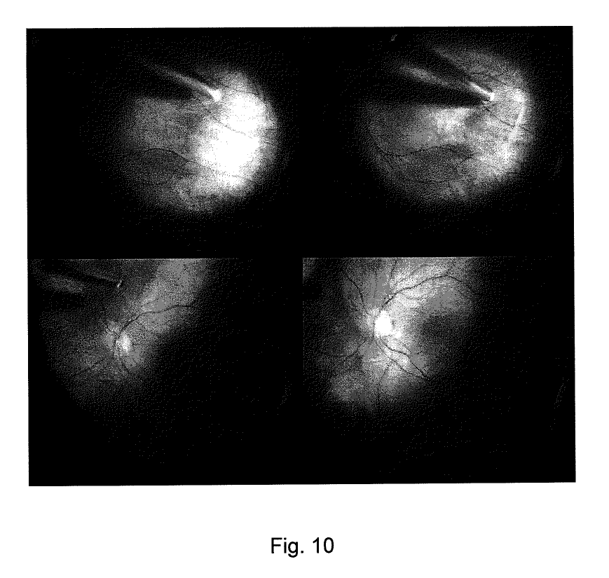

FIG. 10 shows the induction of a posterior vitreous detachment using bromphenol blue. Note the strong adherence of the posterior vitreous cortex and the retinal surface as seen by concentric retinal folds.

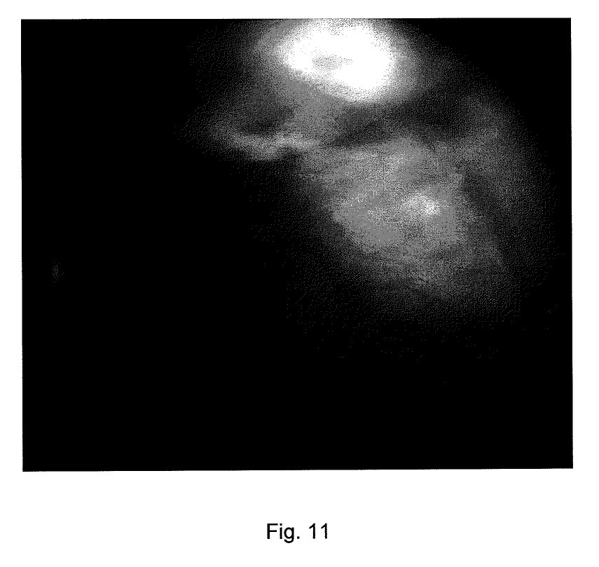

FIG. 11 shows the stained vitreous in the periphery during vitrectomy for retinal detachment.

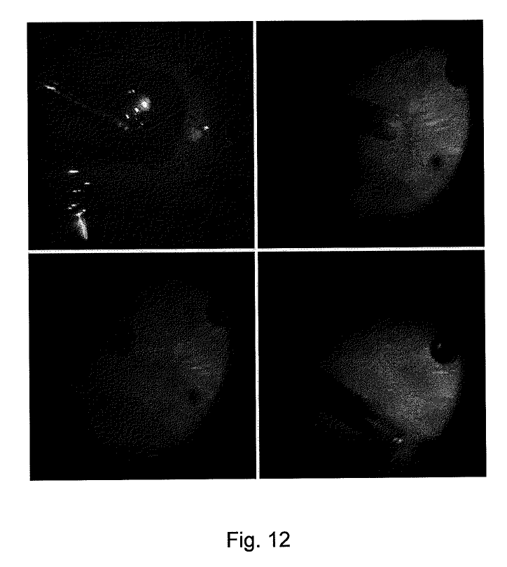

FIG. 12 shows in a further example the peeling of an epiretinal membrane after staining with bromphenol blue.

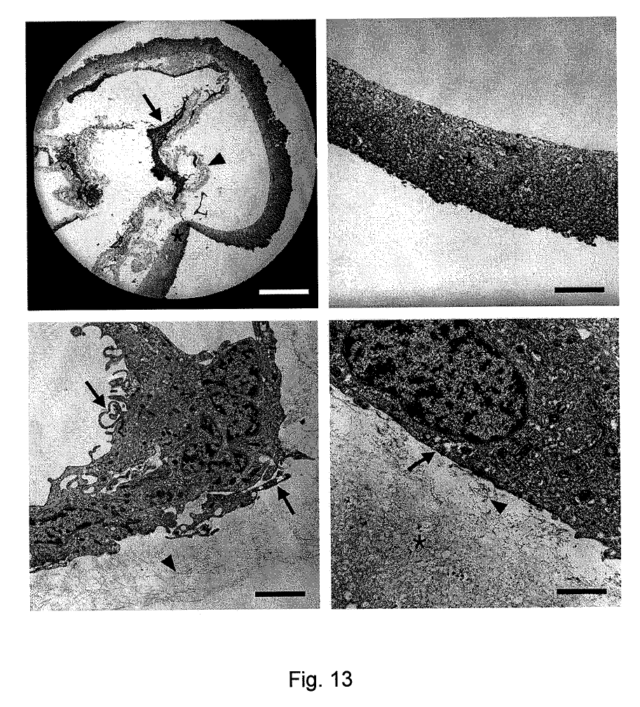

FIG. 13 shows in the fourth example the transmission electron micrographs of the internal limiting membrane (ILM) and epiretinal tissue removed by bromphenol blue-assisted peeling. The ILM was present in most specimens, with native vitreous collagen being interspersed between the ILM and epiretinal cells in many specimens. Newly formed collagen was also seen irregularly distributed between the ILM and cellular elements as well as embedded in areas with cellular proliferation. We observed fibrous astrocytes, fibroblasts, myofibroblasts and RPE cells. Top left, en-bloc peeling of an irregular layer of cells (arrow) and collagen (arrowhead) directly attached to the vitreal side of the ILM (asterisk) (original magnification, ×1000; bar=10.1 μm. Top right, the ILM devoid of cells and collagen (original magnification, ×3600, bar=2.8 μm. Bottom left, densely packed cells with numerous microvillous processes (arrows) on a layer of native vitreous collagen in areas without contact to the ILM (original magnification, ×3600; bar=2.8 μm. Bottom right, fibroblast (arrow) with cellular nucleus and abundant endoplasmatic reticulum at the ILM (asterisk), newly formed collagen (arrowhead) sparsely distributed between fibroblast and ILM (original magnification, ×9500; bar=1.0 μm).

For the following example, methods according to the following section were used.

### Dye Selection and Evaluation of Staining Characteristics

The following four dyes were examined as examples for the inventive dyes in the present study: Light green yellowish SF (LG SF), E68, Bromophenol blue (BPB) and Chicago blue (CB). ICG (Pulsion, Munich, Germany) was used as a reference. All dyes were dissolved and diluted with balanced salt solution (BSS plus; Alcon Laboratories Inc., Fort Worth, Tex.) and concentrations of 1.0, 0.5, 0.2 and 0.05% were obtained. Dry ICG powder was first dissolved with sterile water provided by the manufacturer resulting in a 0.5% solution and then further diluted using BSS plus to a concentration of 0.05%. The dyes were then used to stain lens capsule and epiretinal membranes removed during intraocular surgery. Immediately after removal, the material was placed on a glass slide and covered with a few drops of the dye. After one minute, the dye was carefully removed by irrigation using BSS plus. Lens capsule and epiretinal tissue was then evaluated macroscopically and using light microscopy. The staining effect was subjectively graded as “excellent, good, fair or absent” by one unmasked person (S.P) and photographs were taken.

Additionally, the staining characteristics of the lens capsule in enucleated porcine eyes with a postmortem time of nine hours were evaluated. The dyes were injected into the air-filled anterior chamber and removed by irrigation after one minute. Then, the cornea was removed and a capsulorhexis was performed with a bent needle. All procedures were taped on video.

### Light Absorbing Properties

Light absorption was measured in 0.05% solutions, with BSS plus as a solvent medium. Light absorption was measured instantly after preparation of a stock solution of the dye using a UV/VIS/NIR Spectrometer (Lambda 900, Perkin Elmer) between 200 and 1000 nm. In contrast to ICG the solubility of the other dyes in BSS plus solution is much higher (>0.05% dye).

### Evaluation of Dye Toxicity

### Human RPE Cell Culture

RPE cells from five human donors were obtained from the Eye Bank of the Ludwig-Maximilians-University (Munich, Germany) and were prepared as described in Alge C S, Priglinger S G, Neubauer A S, Kampik A, Zillig M, Bloemendal H, Welge-Lussen U., Retinal pigment epithelium is protected against apoptosis by alphaBcrystallin. Invest Ophthalmol Vis Sci 2002; 43: 3575-82. In brief, whole eyes were thoroughly cleansed in 0.9% NaCl solution, immersed in 5% polyvinyl pyrrolidone iodine, and rinsed again in the sodiumchloride solution. The anterior segment from each donor eye was removed and the posterior poles were examined with the aid of a binocular stereomicroscope to confirm the absence of gross retinal disease. Next, the neural retinas were carefully peeled away from the RPE-choroid-sclera using fine forceps. The eye cup was rinsed with Ca²⁺ and Mg²⁺-free Hank's balanced salt solution, and filled with 0.25% trypsin (GIBCO, Karlsruhe, Germany) for 30 min at 37° C. The trypsin was carefully aspirated and replaced with Dulbecco's modified eagles medium (DMEM, Biochrom, Berlin, Germany) supplemented with 20% fetal calf serum (FCS, Biochrom). Using a pipette, the media was gently agitated, releasing the RPE into the media by avoiding damage to Bruch's membrane. The RPE cell solution was transferred to a 50 ml flask (Falcon, Wiesbaden, Germany) containing 20 ml of DMEM (Biochrom) supplemented with 20% FCS (Biochrom) and maintained at 37° C. and 5% carbon dioxide. Epithelial origin was confirmed by immunohistochemical staining for cytokeratin using a pan-cytokeratin antibody (Sigma). The cells were tested and found free of contaminating macrophages (anti-CD11; Sigma) and endothelial cells (anti-von Willbrand factor, Sigma) (data not shown). After having grown to confluency (100%), primary RPE cells were subcultured and maintained in DMEM (Biochrom) supplemented with 10% FCS (Biochrom) at 37° C. and 5% carbon dioxide. Primary RPE of passage 3-6 and ARPE-19 cells were used for experiments.

ARPE-19 cells, a human retinal pigment epithelial cell line (42), were purchased from ATCC (Manassas, Va., USA) and grown in a 1:1 mixture of Dulbecco's modified Eagles medium and Ham's F12 medium (DMEM/Ham's F12, Biochrom), supplemented with 10% FCS (Biochrom).

### MTT Assay

For exposure of dyes RPE and ARPE-19 cells were kept for 24 hours under serum free conditions. After washing cells three times with PBS, cells were incubated for 10 minutes with 300 μl of BSS plus containing 0.2% or 0.02% of dye, respectively. This rather long exposure time is reasonable as it increases the chance of detecting toxicity, but nevertheless does not mimic the current clinical use of ICG or trypanblue or the likely use of any new dye. The dye was then removed by carefully rinsing cells with BSS plus three times. After 24 hours incubation with serum containing media the cell proliferation assay was performed. RPE cells (p3-6) were seeded in 24-well plates and exposed to two concentrations (0.2 and 0.02%) of dyes. BSS plus alone and H₂O₂, (200 μL/mL) served as controls.

The tetrazolium dye-reduction assay (MTT; 3-[4,5-dimethylthiazol-2-yl]-2,5-diphenyl tetrazolium bromide;) was used to determine cell survival rate. The MTT test was performed as described in the literature by Mosmann T., Rapid calorimetric assay for cellular growth and survival: application to proliferation and cytotoxicity assays. J Immunol Methods 1983; 65: 55-63. The medium was removed, cells were washed with PBS, and 1000 μL/well MTT solution (1.5 mL MTT stock, 2 mg/mL in PBS, plus 28.5 mL DMEM) was added. RPE cells were incubated at 37° C. for 1 hour. The formazan crystals that formed were dissolved by the addition of dimethyl sulfoxide (DMSO; 1000 μL/well). Absorption was measured by a scanning multiwell spectrophotometer at 550 nm (Molecular Probes, Garching, Germany). Results from the wells were expressed as the mean percentage of control proliferation. Experiments were performed in triplicate and repeated three times. RPE and ARPE-19 cells cells of the same passage incubated with BSS without addition of dyes served as control. Statistical comparison between dye concentrations was performed with SPSS (Mann-Whitney-U-Test).

The MTT test as performed in this study is a well established test for the assessment of cell viability, but relies on calorimetric measurement of a blue (550 nm) formazan reaction product. This color overlaps with the absorption spectra of some of the dyes tested. Therefore, control experiments were performed in order to check for potential interferences of residual dye with the assay. Cell monolayers were treated with dyes as per the experiments described above but absorbance readings were performed without prior application of MTT. No differences after dye treatment compared to BSS controls were found. Experiments were performed in triplicate and repeated three times.

### Life-Dead Assay

Confluent RPE and ARPE-19 cells were prepared and treated as described above. Cell viability was quantified based on a two-color fluorescence assay in which the nuclei of nonviable cells appear red because of staining by the membrane-impermeable dye propidium iodide (Sigma), whereas the nuclei of all cells were stained with the membrane-permeable dye Hoechst 33342 (Intergen, Purchase, N.Y.). Confluent cultures of RPE cells growing on coverslips in 24-well tissue culture plates were exposed to 0.2% and 0.02% Light green yellowish SF (LG SF), E68, Bromophenol blue (BPB), Chicago blue (CB) as described for MTT assays. For evaluation of cell viability, cells were washed in PBS and incubated with 2.0 μg/mL propidium iodide and 1.0 μg/mL Hoechst 33342 for 20 minutes at 37° C. Subsequently, cells were analyzed with an epifluorescence microscope (Axioskop; Zeiss, Göttingen, Germany). The labeled nuclei were then counted in fluorescence photomicrographs, and dead cells were expressed as a percentage of total nuclei in the field. The data are based on counts from three experiments performed in duplicate wells, with three to five documented representative fields per well. RPE and ARPE-19 cells of the same passage incubated with BSS without addition of dyes and H₂O₂ (200 μL/mL) served as control.

### Evaluation of Staining Characteristics

The staining effect in removed lens capsule material and ERM varied between the different dyes and dye concentrations applied. Light green yellowish (LGSF) did not stain lens capsule sufficiently using concentrations of 0.5% or less and only a concentration of 1% provided a weak staining of ERM. Lower concentrations were therefore not tested using LGSF. The other dyes revealed excellent to good staining effects both in lens capsule and ERM even at a lower concentration of 0.2%. The grading of the staining effect of each dye and dye concentration is shown in FIG. 6. In porcine eyes, the lens capsule could be stained well with Bromphenol blue (BPB), Chicago blue (CB) and E68, while LGSF provided weak staining effects. In general, the contrast seen in porcine eyes was less pronounced, as the staining effect was evaluated against the background of the clear porcine lens. Intraoperatively, dyes are used to assist capsulorhexis predominantly in mature cataracts, where one should expect a much better contrast.

### Light Absorbing Properties

The light absorbing properties and peaks of maximum absorption of dye concentrations of 0.05% were variable. The long wavelength maximum peak of absorption was in the range 527-655 nm (see FIG. 1). Except Light green SF yellowish (LGSF) no dye showed relevant light absorption between 400 and 500 nm. Absorption maxima beyond 700 nm of any of the investigated dyes were not found.

### Evaluation of Dye Toxicity

### MTT Assay

Compared to BSS plus without addition of any dye serving as a control, the four novel inventive dyes (Light green SF (LG SF); E68; Bromphenol blue (BPB); Chicago blue (CB)) showed no significant impact on cell survival of ARPE-19 cells neither in a concentration of 0.2 nor of 0.02% (see FIG. 2). Additionally, no influence on cell survival of primary RPE cells was observed after exposition to LGSF, E68, BPB and CB at concentrations of 0.2 and 0.02% (FIG. 3). The differences between both concentrations were statistically significant only for Light green SF (LG SF, p≦0.05) and Indocyanine green (ICG, p≦0.05) in primary RPE cells.

### Life-Dead Assay

When the viability of RPE cells was tested by labeling of the nuclei of nonviable cells with propidium iodide 24 hours after treatment of cells, two dyes (Light green yellowish (LGSF) and Chicago blue (CB)) were identified to significantly affect cell viability compared to controls treated with BSS plus alone. After treatment with CB, this effect was seen both in cultures of ARPE-19 and primary RPE cells in concentrations of 0.2% and 0.02%. However, in comparison to the 0.2% dye solution, 0.02% LG SF appeared to be far less toxic. E68, Bromophenol blue (BPB) and BSS plus (control) did not affect cell survival.

### EXAMPLE 1

### Staining Characteristics of the Vitreous (Corpus Vitreum)

Five dyes (Light green SF yellowish (LG SF); E68; Bromophenol blue (BPB); Chicago blue (CB); Rhodamine 6G) were included in this investigation. All dyes were dissolved and diluted using balanced salt solution (BSS plus; Alcon Laboratories Inc., Fort Worth, Tex).

Experiments were performed in animals (porcine eyes) and humans after approval of the local ethics committee had been obtained. All experiments were performed in compliance with the Declaration of Helsinki.

### Surgical Procedure in Porcine Eyes (LG SF; E68; BPB, CB; Rhodamine 6G):

Ophthalmic surgery consisted of a standard pars plana vitrectomy. First, the transparent vitreous was removed as completely as possible. Then fluid air exchange was performed in order to avoid an uncontrolled distribution of the dye in the vitreous cavity. The dye was then injected into the air filled globe. After a period of one minute, the dye was washed out by irrigation with BSS. This was followed by an injection of the dye in concentrations from 0.2% to 2%. The staining of the vitreous was graded by two examiners who also performed the surgical procedures.

The staining effect was graded as shown in the following table:

dye concentration

| | 0.2% | 2.0% |
|---|---|---|
| BPB | good | strong |
| LGSF | strong | strong |
| E68 | weak | strong |
| CB | weak | strong |
| Rhodamine 6G | good | strong |

### Surgical Procedure in Humans Using BPB:

All patients (n=9) gave their written informed consent before surgery. Surgery was performed to treat tractive maculopathies such as macular pucker and macular hole, where increased adherence of the vitrous and the retinal surface has been described to significantly contribute to the pathogenesis of the disease. A standard three port pars plana vitrectomy was performed. First, the transparent vitreous was removed as thoroughly as possible using the vitrectomy probe. Then fluid air exchange was performed and a few drops of the dye (BPB, 0.2%) were injected. After one minute, the dye was washed out by irrigation. A bluish staining of vitrous remnants adherent to the retinal surface was observed. Due to the visualization of the vitreous, a complete removal of the vitrous could be performed without complications.

### Conclusion

We were able to show, that the dyes investigated stain the vitreous gel, which is usually transparent. This will be of relevance during surgical procedures were a thorough and complete removal of the vitreous is crucial, in order to relief tractional forces at the retinal level. This is the case during surgical procedures for the treatment of retinal detachment and tractive maculopathies as well as proliferative vitreoretinopathy or diabetic retinopathy.

### EXAMPLE 2

### Example for Capsulorhexis Using Bromphenol Blue.

Capsulorhexis in patients with mature, white cataracts can be challenging due to absence of the red fundus reflex. Therefore, dye-enhanced cataract surgery has become the preferred method to allow the surgeon to perform a controlled rhexis of the anterior capsule in these cases. Several dyes have been suggested and used for this purpose, among them fluorescein, indocyanine green (ICG), and trypan blue The latter is the most frequently used blue dye at present and the concentrations used to stain the lens capsule vary between 0.0125% to 0.1%. Trypan blue has a good safety profile in the long term.

In previous experimental studies, we have investigated a number of vital dyes in-vivo and in-vitro in order to evaluate their staining characteristics and safety (C. Haritoglou, A. Yu, W. Freyer, S. G. Priglinger, C. Alge, K. Eibl, C. A. May, U. Welge-Luessen, A. Kampik, “An evaluation of novel vital dyes for intraocular surgery”, Invest Ophthalmol Vis Sci 46 (2005), pp. 3315-22; F. Schuettauf, C. Haritoglou, C. A. May, R. Rejdak, A. Mankowska, W. Freyer, K. Eibl, E. Zrenner, A. Kampik, S. Thaler, “Administration of novel dyes for intraocular surgery: an in vivo toxicity animal study”, Invest Ophthalmol Vis Sci 47 (2006), pp. 3573-8; C. Haritoglou, R. Tadayoni, C. A. May, C. A. Gass, W. Freyer, S. G. Priglinger, A. Kampik, “Short-term in vivo evaluation of novel vital dyes for intraocular surgery”, Retina 26 (2006), pp. 673-8). As a result from these studies, bromphenol blue was identified not only to strongly stain the anterior lens capsule, but also to be safe for intraocular application. Prior to the application in humans, the toxicity of bromphenol blue had been thoroughly investigated in experimental settings including ex-vivo cell culture models and in-vivo animal studies with intraocular exposure times of up to seven days (C. Haritoglou, A. Yu, W. Freyer, S. G. Priglinger, C. Alge, K. Eibl, C. A. May, U. Welge-Luessen, A. Kampik, “An evaluation of novel vital dyes for intraocular surgery”, Invest Ophthalmol Vis Sci 46 (2005), pp. 3315-22; F. Schuettauf, C. Haritoglou, C. A. May, R. Rejdak, A. Mankowska, W. Freyer, K. Eibl, E. Zrenner, A. Kampik, S. Thaler, “Administration of novel dyes for intraocular surgery: an in vivo toxicity animal study”, Invest Ophthalmol Vis Sci 47 (2006), pp. 3573-8; C. Haritoglou, R. Tadayoni, C. A. May, C. A. Gass, W. Freyer, S. G. Priglinger, A. Kampik, “Short-term in vivo evaluation of novel vital dyes for intraocular surgery”, Retina 26 (2006), pp. 673-8). Clinical examinations of the animals during the period of dye exposure of seven days did not reveal any signs of inflammatory responses or toxicity in the anterior segment of the eye such as corneal edema even after injection of higher dye concentrations and no histological abnormalities were seen following enucleation (C. Haritoglou, A. Yu, W. Freyer, S. G. Priglinger, C. Alge, K. Eibl, C. A. May, U. Welge-Luessen, A. Kampik, “An evaluation of novel vital dyes for intraocular surgery”, Invest Ophthalmol Vis Sci 46 (2005), pp. 3315-22). The data obtained from these toxicity studies underlined the high biocompatibility of bromphenol blue. Bromphenol blue (C₁₉H₁₀Br₄O₅S, FW 670) has been used in the past as a vital stain to probe the blood-brain barrier and as a protein stain and as a pH indicator. In the present example, we describe experience using bromphenol blue to stain the human lens capsule in patients with white cataracts. This investigation was approved by the local ethics committee and the Institutional Review Board (IRB). Written informed consent was obtained from all patients prior to surgery.

Cataract formation was due to ocular trauma (n=2), uveitis (n=1) and unknown cause (n=2). Mean age was 59 years. All patients underwent a complete clinical examination before surgery. B-scan ultrasound of the globe was performed to rule out intraocular pathologies such as tumors or retinal detachments. Bromphenol blue powder was dissolved and diluted using BSS plus (balanced salt solution; Alcon Laboratories Inc., Fort Worth, Tex, USA) and sterilized using a 0.22 μm syringe filter. We used dye concentrations of approximately 0.1% (310 mOsm, pH 7.6). Five consecutive patients were included into this preliminary series of patients, three female and two male. Visual acuity was 20/400 or below before surgery and increased to 20/50 (mean) postoperatively. The dye was either injected into the air-filled (n=3) anterior chamber and carefully removed by injection of viscoelastic material (FIG. 7) immediately after injection or injected after having filled the anterior chamber with viscoelastic material (n=2) without prior air injection. In the latter case, the dye was evenly and gently distributed on the lens surface using the cannula and excessive dye was removed after completion of capsulorhexis (approximately one minute after application).

Both techniques allowed for a predictable and uniform staining of the anterior lens capsule due to the direct contact of the dye with the capsule. A circular capsulorhexis could be performed in four cases using a bent needle (FIG. 7). In one patient, the lens capsule was very rigid and had to be opened using an intraocular forceps. An excellent contrast between the white lens and the stained lens capsule was noted in all patients. The dye did not penetrate the lens capsule. There was no unwanted staining of other tissues such as the iris or the corneal endothelium. During surgery, a staining of the limbal incision through which the dye had been injected was observed, which disappeared shortly after dye application. One day postoperatively, no residual dye was detected on the remaining rim of the anterior lens capsule or any other ocular tissue. All patients were followed up to five months postoperatively. There were no complications noted such as corneal edema, signs of inflammation or relevant decrease of corneal endothelial cells.

Based on our present observations, bromphenol blue can be used in anterior segment surgery without concerns.

### EXAMPLE 3

### Example for Vitrectomy Using Bromphenol Blue

Besides staining of an ERM or the ILM, a visualization of the vitreous itself has become a field of interest among vitreoretinal surgeons. The vitreous may function as a scaffold for fibrovascular proliferation and interactions of vitreous collagen fibers and the innermost retina at the vitreoretinal interface represent the underlying mechanism of action for tractional vitreoretinal diseases. Therefore, the thorough removal of the vitreous and the posterior hyaloid membrane is an important goal during vitreoretinal procedures to treat macular holes, vitreoretinal traction syndrome, retinal detachment and many other conditions.

As a consequence, the visualization of the vitreous seems to be an important aspect of “chromovitrectomy”, a new field in vitreoretinal surgery. In the present study, we describe the staining of the vitreous and the posterior hyaloid using bromphenol blue in humans.

This study was approved by the local ethics committee and Institutional Review Board (IRB) and written informed consent was obtained from all patients. Six consecutive patients with idiopathic macular hole, 3 males and 3 females with a mean age of 68 were included. Holes were graded as stage 2 (n=3), stage 3 (n=2) and stage 4 (n=1). The dye was also used in four patients with retinal detachments, 2 females and 2 males with a mean age of 57 years.

Pre- and postoperatively, all patients underwent a complete clinical examination including measurement of best corrected visual acuity (VA), slit lamp examination, tonometry, and funduscopy using a 78 diopter lens (Volk Optical, Mentor, Ohio, USA). In macular hole patients, we additionally performed optical coherence tomography (OCT) (Stratus-OCT, Carl Zeiss Meditec, Jena, Germany), Goldmann perimetry, multifocal electroretinogram (ERG) and fundus photographs. Patients were seen one day before surgery and then in six weeks intervals. Postoperatively, ERG and Goldmann perimetry were performed at the six weeks follow-up visit and were not repeated if unremarkable.

Bromphenol blue powder was dissolved and diluted using BSS plus and sterilized using a 0.22 μm syringe filter. A final dye concentration of 0.2% was then injected into the eye. Vitrectomy first consisted of a core vitrectomy, followed by vitreous removal in the periphery in the area of the sclerotomy sites by indentation of the globe using a squint hook. Then an attempt was made to induce a posterior vitreous detachment by applying suction at the optic nerve head.

The dye was applied as follows: During pars plana vitrectomy for a macular hole a fluid air-exchange was performed prior to dye injection to achieve a maximum dye concentration on the retinal surface. Then, a few drops of the dye were injected over the posterior pole and the globe was gently moved to allow for an adequate dye distribution (FIG. 8A). This was followed by removal of excessive dye by irrigation after one minute. Then epiretinal tissue was removed if present and an attempt to peel the ILM using an end-gripping forceps was made in selected cases. Surgery was completed by fluid-air exchange and intraocular gas tamponade (hexafluoroethane (C₂F₆)) as previously described (Haritoglou C., Gandorfer A., Gass C. A., et al., “Indocyanine green-assisted peeling of the internal limiting membrane in macular hole surgery affects visual outcome: a clinicopathologic correlation”, Am J. Ophthalmol 2002; 134: 836-41). During surgery for retinal detachment the dye was applied after induction of a PVD and injection of approximately 1.5 ml heavy liquid (PFCL, perfluorocarbon, Fluoron GmbH, Neu-Ulm, Germany) and fluid-air exchange without removing PFCL (FIG. 8B). Using this approach, we obtained not only a higher dye concentration in the anterior segment of the eye as the dye can not be further diluted by PFCL, but we could also prevent an uncontrolled distribution especially in the subretinal space and excessive contact with the lens capsule, which may result in an unwanted staining effect. In addition we obtained a staining of the vitreous in the periphery as intended. C₂F₆ was used as an intraocular tamponade at the end of surgery. The staining characteristics were then evaluated by the surgeon.

We included 6 patients with idiopathic macular holes and 4 patients with retinal detachment. In patients undergoing macular hole surgery, the following staining pattern using bromphenol blue was observed: In two patients a very thin layer of vitreous cortex firmly adherent to the retinal surface was visualized, although the surgeon had been confident of having induced a mechanical posterior vitreous detachment (PVD) prior to dye injection (FIG. 9). In one patient, the surgeon could not create a complete PVD before dye application, but was able to complete this maneuver due to a visualization of the vitreous cortex following dye injection. Therefore, in all of these cases, the visualization of the remnant and attached posterior hyaloid helped to create a complete PVD in a second step (FIG. 10). Strong adherences to the retinal surface and especially the macular area and the posterior pole became apparent due to the formation of retinal folds at the border of attached and detached posterior hyaloid (FIG. 10). Following complete PVD a relief of tractional forces in the area of the macular hole was observed and ILM peeling was therefore only performed in one of these cases with the size of the hole having been larger that 400 μm in diameter. In two of these patients ILM peeling was not performed with respect to the small size of the hole and the observation that the hole appeared to be closed by PVD alone. In the other three cases, no staining of the retinal surface or the ILM was noted following dye injection, indicating that there was a complete PVD and no epiretinal membrane present. Staining of remnant vitreous in the periphery at the vitreous base could be seen in all cases.

All macular holes could be closed successfully with a second operation having been necessary in one case. Over all patients VA increased from 20/100 preoperatively to 20/40 postoperatively. No loss of VA was seen. In one patient, who required two surgical approaches to close the hole, VA remained unchanged. We found no affection of the amplitudes in postoperative ERG examinations nor did we observe peripheral visual field defects indicating potential dye related toxicity.

During surgery for retinal detachment, bromphenol blue helped to identify peripheral remnants of vitreous in the area of the vitreous base and the retinal breaks (FIG. 11). The visualization greatly facilitated the removal of the vitreous and made surgery more reliable and safe. A reattachment of the retina could be achieved in all cases

In the present report, we describe experiences using bromphenol blue for a better visualization of the vitreous and the posterior hyaloid membrane during pars plana vitrectomy for macular hole and retinal detachment.

Bromphenol blue was very useful to stain the adherent posterior hyaloid membrane and visualize interactions in terms of tractional forces of the vitreous on the retinal surface and in the macular area in macular hole patients with incomplete PVD.

### EXAMPLE 4

### Example for Vitreoretinal Surgery Using Bromphenol Blue

Vital dyes potentially facilitate vitreoretinal surgery by visualising nearly transparent structures such as the internal limiting membrane (ILM) and epiretinal membranes (ERM). Especially for surgeons at the beginning of their learning curve, the use of dyes may help to reduce the risk of mechanical trauma and damage of underlying structures, such as the nerve fibre layer, and allow for a more complete removal of the target structure. Two dyes are currently available for this intraocular application: indocyanine green (ICG) and trypan blue. Whereas ICG has been shown to selectively stain the ILM, trypan blue is mainly used to visualise ERM.

ICG became the subject of ongoing discussion as clinical and experimental data suggested dye-related toxicity, leading to less favourable functional outcome after macular surgery. As the underlying mechanisms of action as well as the safety margins of ICG are not completely understood so far, the applicability of ICG seems to be limited. Although no significant clinical adverse events have been reported for trypan blue in humans, chronic and acute toxic effects have been seen in animals and cell culture models.

In the present example, we describe the use of bromphenylblue during vitreoretinal surgery for tractive maculopathies such as macular holes and macular pucker.

The study was approved by the local ethics committee and Institutional Review Board, and written informed consent was obtained from all patients. Thirteen patients with macular pucker, seven males and six females with a mean age of 65, were included in the present study.

Preoperatively and postoperatively, patients underwent a complete clinical examination including measurement of best corrected visual acuity (VA), slit-lamp examination, tonometry, funduscopy using a 78 diopter lens (Volk Optical, Mentor, Ohio, USA), fluorescein angiography, OCT (Stratus), Goldmann perimetry, multifocal ERG and fundus photograph. Patients were seen one day before surgery and then in six-week intervals. Postoperatively, ERG and Goldmann perimetry were performed at the six-week follow-up visit and were not repeated if unremarkable.

Bromphenol blue was dissolved and diluted using BSS plus and sterilised using a 0.22 μm syringe filter and dye concentrations of 0.2% were then injected into the eye.

Vitrectomy consisted of a standard three-port pars plana vitrectomy as described in previous reports (Haritoglou C., Gandorfer A., Gass C. A., et al., “Indocyanine green-assisted peeling of the internal limiting membrane in macular hole surgery affects visual outcome: a clinicopathologic correlation”, Am J. Ophthalmol 2002; 134:836-41; Haritoglou C., Gandorfer A., Gass C. A., et al.: “The effect of indocyanine-green on functional outcome of macular pucker surgery, Am J. Ophthalmol 2003; 135:328-37). Before injection of the dye, a fluid air-exchange was performed to avoid an uncontrolled dye distribution. Then, a few drops of the dye were applied over the macular area. After a period of one minute, the dye was completely washed out by irrigation. The staining characteristics were then evaluated by the surgeon and an additional examiner (CH). This was followed by removal of epiretinal tissue and the ILM using an end-gripping forceps. After the removal of epiretinal tissue, no second dye injection was performed in this series of patients.

All epiretinal tissue removed during surgery was harvested and immediately prepared for ultrastructural analysis. Ultrathin sections were obtained from all specimens, contrasted with uranyl acetate and lead citrate, and analysed using a Zeiss EM 9 electron microscope (Zeiss Jena, Germany). Histological evaluation of specimens was performed by one of the coauthors who did not observe the surgical procedure and had no access to other patient data.

We included 12 patients with idiopathic macular pucker and one patient with ERM formation in association with proliferative diabetic retinopathy. Patients were operated on using a dye concentration of 0.2% (FIG. 12). Staining effect varied depending on the thickness of the ERM. Less staining was noted in cases where it appeared difficult to remove an ERM, suggesting that the pathological alterations were more pronounced within the retina and not on the retinal surface. We did not observe sufficient staining of the ILM in our patients with ERM. Staining of remnant vitreous in the periphery at the vitreous base could he seen in all cases.

Overall patients VA increased from 0.16 preoperatively to 0.32 postoperatively. No loss of VA was seen. No dye-related complications were observed in any functional test performed. We found no affection of the amplitudes in postoperative ERG examinations nor did we observe peripheral visual field defects attributable to the dye.

Epiretinal membranes of 8 patients following vitrectomy were evaluated using microscopy. The histological evaluation focused on the appearance of the epiretinal cellular layers and the amount of cellular debris on the retinal surface of the ILM (FIG. 13). Epiretinal cells all had maintained their cellular integrity. Small cellular fragments at the retinal surface of the ILM were frequently noted.

With respect to reports on adverse effects following the use of ICG, which is mainly used to stain the ILM, but also for trypan blue, it seems reasonable to look for potential new dyes with a better safety profile and reliable safety margins for intraocular application.

We observed that the staining effects of bromphenol blue varied with less staining noted in cases where hardly any tissue could be peeled off. Otherwise, a nice demarcation of the membrane was observed. Bromphenol blue might serve as a diagnostic tool intraoperatively, helping to avoid excessive peeling in cases of predominant intraretinal abnormalities.

We also observed a strong staining of vitreous remnants at the vitreous base in all cases. This seems to be relevant as a complete removal of the vitreous is crucial for surgical success in many conditions.

Vitreous staining is a very helpful tool, especially for less experienced surgeons, during surgery for retinal detachment, where a thorough removal of vitreous in the area of the retinal break is important, or tractive retinopathy in proliferative diabetic retinopathy. In addition, the detection and complete dye-assisted removal of vitreous remnants on the retinal surface may provide enough relief of tractional forces, for example in smaller (stage II) macular holes, and ILM peeling might therefore not always be necessary.

Histological evaluations of ERM removed during surgery showed morphological features which were in line with previous reports in the literature. Of note, we did not observe a disruption of epiretinal cells or great amounts of cellular debris on the retinal surface of the ILM as a sign of dye-related toxicity.

In summary, bromphenol blue provided good staining properties in epiretinal membranes and vitreous and showed a very good safety profile intraoperatively and during follow-up.

### SUMMARY

The use of vital dyes to intraoperatively stain ocular tissue such as the lens capsule, the vitreous or epiretinal membranes or the ILM potentially facilitate ophthalmic surgery. The introduction especially of ICG to assist macular surgery was initially met with great enthusiasm in the ophthalmic community as it appeared to make intraocular surgery safer and more controllable by visualizing and facilitating the removal of the ILM during surgery for tractive maculopathies such as macular pucker or macular holes.

Additionally, staining of the vitreoretinal interface could open the door also for the less experienced surgeon to follow the principle of ILM removal in macular surgery. However, as there are observations indicating potential dye related toxicity under yet not completely understood circumstances, the use of ICG has become a controversial subject among surgeons. The question whether ICG should be considered a “toxic adjunct” is currently under investigation. No in-vivo or in-vitro safety studies concerning the intraocular use of ICG preceded the clinical, intraocular application of ICG.

Given the known chemical properties of ICG and the current debate on potential toxic effects of ICG after intraocular application, the following considerations might be of interest when choosing and evaluating novel dyes:
a) The dye should exhibit a large absorption coefficient. Large extinction coefficients allow for the injection a significantly lower amount of the dye. As a consequence the formation of aggregates is suppressed at low dye concentrations.
b) The dye should show a high solubility in water and BSS (which is used as an irrigation solution during surgery), but should not show a concentration dependence. In most cases this effect is accompanied by a dramatic shift of the absorption maximum and/or with the appearance of new absorption bands.
c) The material should have a high photochemical stability. That means irreversible photoreaction in the first excited singlet state. S1 or triplet state T1, as described for the photosensitizer ICG, should be avoided.
d) The triplet quantum yield should be as low as possible avoiding singlet oxygen formation that can lead to an irreversible oxidation of the ILM / tissue or to photodegradation of the dye itself. On the other hand the lifetime of singlet oxygen both in water and in the solid state is very short.
e) The staining material should have a minimal dark toxicity.
f) The dye should exhibit very good adsorption properties towards the target tissue.

It is evident from the above brief description that all requirements can hardly be met and compromises should be found.

With the present examples, different dyes of both cationic and anionic character from different dye classes have been shown to be highly suitable for ophthalmic surgery according to the above criteria. These dyes have large absorption coefficients (5×10⁴−20×10⁵ L mol⁻¹ cm⁻¹). All compounds show a high solubility in water and BSS solution. Their photostability and dark stability in water or BSS solution are in some cases much better than the one of ICG (data not shown). The position of the long wavelength maximum of Light green SF yellowish, E68, Bromophenol blue, Chicago blue is not influenced by the dye concentration (measured up to a content of 0.05% dye).

1. Method of staining the internal limiting membrane (membrana limitans interna, ILM), the vitreous (corpus vitreum), epiretinal membranes (ERM) and/or the lense capsule of the eye, comprising administering to the eye at least one of a fluorescent dye, Lightgreen SF, Copper-phtalocyanine-tetrasulfonic acid tetrasodium salt, E68, Bromophenolblue and Chicagoblue.

2. Method according to claim 1, wherein at least one of a fluorescent dye, Lightgreen SF, Copper phtalocyanine-tetrasulfonic acid tetrasodium salt, E68, Bromophenolblue and Chicagoblue is administered by intraocular injection.

3. Method according to claim 1, wherein at least one of a fluorescent dye, Lightgreen SF, Copper phtalocyanine-tetrasulfonic acid tetrasodium salt, E68, Bromophenolblue and Chicagoblue is administered to the internal limiting membrane and/or the lense capsule.

4. Method according to claim 1, wherein at least one of a fluorescent dye, Lightgreen SF, Copper phtalocyanine-tetrasulfonic acid tetrasodium salt, E68, Bromophenolblue and Chicagoblue is administered to a mammal.

5. Method according to claim 4, wherein the mammal is a human.

6. Method according to claim 1, wherein the fluorescent dye is Rhodamine.

7. Medicament comprising at least one of a fluorescent dye, Lightgreen SF, Copper phtalocyanine-tetrasulfonic acid tetrasodium salt, E68, Bromophenolblue and Chicagoblue.

8. Medicament according to claim 7, wherein the fluorescent dye is Rhodamine.

9. The method of claim 1, wherein the administering is carried out in ophthalmic surgery.

10. The method of claim 9, wherein the ophthalmic surgery is a macular surgery, vitrectomy and/or cataract surgery.

11. The method according to claim 9, wherein the fluorescent dye is Rhodamine.

12. The method according to claim 9, wherein the ophthalmic surgery is of a mammal.

13. The method according to claim 12, wherein the mammal is a human.
